(19) United States
(12) Patent Application Publication
(10) Pub. No.: US 2023/0398275 A1
(43) Pub. Date: Dec. 14, 2023

(54) VALVE DEVICE, EFFLUENT BAG AND METHODS

(71) Applicant: Fresenius Medical Care Deutschland GmbH

(72) Inventor: Juergen Klewinghaus, Oberursel (DE)

(21) Appl. No.: 18/034,201

(22) PCT Filed: Oct. 27, 2021
(86) PCT No.: PCT/EP2021/079863

§ 371 Date: Apr. 27, 2023

(30) Foreign Application Priority Data
Oct. 28, 2020 (DE) 102020128296.6

(51) Int. Cl. A61M 1/16; A61M 1/34; A61M 1/36

(52) U.S. Cl. CPC A61M 1/1649; A61M 1/3413; A61M 1/3621; A61M 2205/07

(57) ABSTRACT

The present disclosure relates to a valve device for connecting to an outlet tap associated with an effluent outlet opening of an effluent bag. The valve device includes an actuator and is arranged in or on an effluent outlet line or an attachment therefor. The actuator of the outlet tap can be brought into two different settings, which can block or allow the flow from the effluent outlet opening along the effluent outlet line. The valve device has a holding section, an insert section and a blocking element which can be switched between positions by which the second fluid line can be acted on to interrupt a flow along the second fluid line and to insulate an electrically conductive liquid column in the second fluid line in an electrically insulating manner or to allow a flow.

### CROSS-REFERENCE TO RELATED APPLICATIONS

The present application is the national stage entry of International Patent Application No. PCT/EP2021/079863, filed Oct. 27, 2021, and claims priority to DE 102020128296.6, filed in the Federal Republic of Germany on Oct. 28, 2020, the disclosures of which are expressly incorporated herein in their entirety.

### TECHNICAL FILED

The present disclosure relates to a valve device as described herein and an effluent bag as described herein. It also relates to a set as described herein and a method as described herein for preparing an effluent bag and a method as described herein for emptying the effluent bag. Furthermore, the present disclosure relates to a blood treatment apparatus as described herein.

### BACKGROUND

Extracorporeal blood treatment is known from practice. Whereby the patient's blood is taken and fed extracorporeally along a blood circuit and through, for example, a blood filter. The blood filter includes a blood chamber through which blood is guided, and a dialysis liquid chamber, through which dialysis liquid is guided. Both chambers are separated from each other by a semi-permeable membrane. Blood and dialysis liquid are mostly guided through the blood filter by the counter current principle. The blood is purified in the blood filter, and on exiting the blood filter, the dialysis liquid, from now on referred to as dialysate, is regarded as used and is discarded. In addition to the dialysate, the fluid to be discarded also includes filtrate, which includes water that has been withdrawn from the blood in the blood filter. Filtrate and dialysate will be referred to individually or collectively in the following simply as effluent.

The effluent is fed to an effluent bag via an effluent inlet line and is initially stored therein. After completion of the blood treatment, or in bag draining intervals during the blood treatment (intervals in which the effluent bag is emptied), the effluent is discarded from the effluent bag into a washbasin or a drain, over which it is held.

In some aspects, the present disclosure provides a valve device for use with an effluent bag used in blood treatment and a further effluent bag for use in blood treatment.

Furthermore, a set with a valve device as described herein, a method for preparing the effluent bag for an upcoming blood treatment, a method for emptying the effluent bag, and a blood treatment apparatus should be specified.

### SUMMARY

The present disclosure relates to a valve device, designed and provided to be connected to an outlet tap which is assigned or connected to an effluent outlet opening of an effluent bag for receiving effluent resulting from a blood treatment. Such an outlet tap, to which the valve device can be connected for its intended use, is usually already arranged in or on an effluent outlet line connected to the effluent outlet opening or an attachment for such an effluent outlet line, via which the effluent present in the effluent bag can run off in whole or in part in order to be discarded. Such an outlet tap has an actuator which may be brought into first setting, in which the flow from the effluent outlet opening along the effluent outlet line is blocked, and in a second setting, in which the flow from the effluent outlet opening is to be released and out of the effluent bag along the effluent outlet line.

The valve device includes a holding section (alternatively: connecting section) via which the valve device, preferably in a form-fitting and/or force-fitting manner, is held at or on the outlet tap or can be held or connected to the outlet tap.

The valve device further includes an insert section which serves to insert a second fluid line or a section thereof into the valve device or a section thereof. The second fluid line can also be referred to herein as a connecting line or “extension” of an effluent inlet line, which is discussed in more detail below, via which effluent coming from the dialysis fluid chamber of the treatment apparatus is fed or conveyed into the effluent bag.

The valve device further includes an blocking element, which can be switched between a first and second position. Here, the blocking element is arranged in the first position to act directly or indirectly on the second fluid line to block flow along the second fluid line and preferably, when in use, to interrupt an electrically conductive liquid column in the second fluid line in an electrically insulating manner and, in the second position, to allow flow along the second fluid line.

The present disclosure also relates to an effluent bag which is designed to hold effluent resulting from a blood treatment and which also has a valve device as described herein.

The effluent bag described herein hereby includes a, preferably closable, effluent inlet opening and a, preferably closable, effluent outlet opening which is separate from the inlet opening. The effluent inlet opening and the effluent outlet opening are used to connect the inside of the effluent bag to the outside of the effluent bag for the supply or removal of effluent to or from the effluent bag.

The effluent bag described herein further includes the outlet tap connected or to be connected to the effluent outlet opening, which includes an actuator and is arranged in or on an effluent outlet line, wherein the actuator of the outlet tap could be brought into a first setting of the outlet tap, in which the flow from the effluent outlet opening along the effluent outlet line is blocked, and into a second setting in which the flow from the effluent outlet opening along the effluent outlet line and out of the effluent bag is released.

A set described herein includes a valve device described herein and a second fluid line for insertion into the insert section of the valve device described herein.

The method described herein serves to prepare an effluent bag for receiving the effluent resulting from a blood treatment.

This method encompasses providing an effluent bag as described herein or a set as described herein.

Furthermore, the method encompasses connecting the second fluid line to both the effluent inlet line and the effluent inlet opening. Suitable connectors are provided for this at the free ends of the second fluid line of the effluent bag or the valve device of the set.

The insertion of the second fluid line into the insert section of the valve device, if not already done, is also encompassed.

Finally, the present disclosure describes connecting the valve device to the outlet tap via the holding section or connecting section of the valve device.

The method described herein for emptying an effluent bag includes providing an effluent bag as described herein or a set as described herein, preferably each prepared according to the above-mentioned method for preparing the effluent bag.

Furthermore, the method for emptying an effluent bag includes the actuation of a switching element of the valve device described herein in such a way that a fluid connection is established between the interior of the effluent bag and the interior of the effluent outlet line.

All advantages achievable with the method described herein can also be achieved in certain embodiments undiminished with the devices described herein, and vice versa.

The blood treatment apparatus described herein is connected to an effluent bag as described herein or to a set as described herein, the effluent bag described herein or the effluent bag of the set described herein preferably each being prepared according to the method described herein for preparing the effluent bag.

Embodiments may include several or some or all of the following features in any combination, unless the person skilled in the art recognizes their combination as technically impossible.

In all of the following statements, the use of the expression “may be” and “may have” etc. is synonymous to “is preferably” or “has preferably,” etc. respectively, and is intended to illustrate embodiments according to the present disclosure.

Whenever numerical words are mentioned herein, the person skilled in the art shall recognize or understand them as indications of a numerical lower limit. Unless it leads the person skilled in the art to an evident contradiction, the person skilled in the art shall comprehend the specification for example of “one” as encompassing “at least one”. This understanding is also equally encompassed by the present disclosure as the interpretation that a numeric word, for example, “one” may alternatively mean “exactly one”, wherever this is evidently technically possible for the person skilled in the art. Both understandings are encompassed by the present disclosure and apply herein to all used numerical words.

Whenever an embodiment is mentioned herein, it represents an exemplary embodiment according to the present disclosure.

When it is disclosed herein that the subject-matter described herein includes one or several features in a certain embodiment, it is also respectively disclosed herein that the subject-matter described herein explicitly does not include this or these features in other embodiments, e.g. in the sense of a disclaimer. Therefore, for every embodiment mentioned herein it applies that the converse embodiment, e.g., formulated as negation, is also disclosed.

Embodiments may include one or all of the aforementioned and/or following features in any technically possible combination.

In some embodiments, the outlet tap has a through-opening or a through-lumen for leading a section of an effluent outlet line or an effluent flowing in the effluent outlet line through the outlet tap. The outlet tap may have a grip section which, when used as intended, serves to manually transfer an actuator of the outlet tap from its first setting to its second setting, and vice versa.

In some embodiments of the valve device described herein, the valve device and/or the outlet tap are not in active or fluid communication with a pneumatic drive device.

In several embodiments of the valve device described herein, the blocking element is arranged so that as the actuator of the outlet tap is being moved into the first setting, the blocking element is forcibly being moved into the second position, and then as the actuator is being moved into the second setting the blocking element is forcibly being moved into the first position.

In some embodiments the actuator of the outlet tap is arranged such that as the blocking element is being moved into the first position, the actuator is forcibly being moved into the second setting, and then as the blocking element is being moved into the second position, the actuator is forcibly being moved into the first setting.

In several embodiments the blocking element is arranged such that, when the actuator of the outlet tap is moved into the second setting, the blocking element is already forced to assume or to maintain a position in which the flow along the second fluid line is or remains prevented, and in which, during use, preferably an electrically conductive liquid column in the second fluid line is interrupted in an electrically insulating manner or the fluid line is or remains blocked before the actuator assumes the second setting or firstly allows a flow through the effluent outlet line.

In some embodiments, the holding section of the valve device described herein has a holder, a rotary bolt, and a cover. In this case, the holder has the insert section for the second fluid line, which can be closed using the rotary bolt, which serves as a safeguard against the second fluid line being removed from the valve device.

Here, the cover can be provided on the holder in such a way that the cover is movable between a first, open cover position for inserting or removing the outlet tap and possibly also a section of the first fluid line into or out of the holding section or the holder, and a second, closed cover position for force-fitting and/or form-fitting holding of the outlet tap in the holding section.

In several embodiments, the holding section has a holder (e.g., the one previously mentioned) for receiving the outlet tap or a portion thereof and a cover (e.g., the one previously mentioned), wherein the holder and/or cover are designed with a snap or click system. The snap or click system is used for their mutual or common connection and/or for at least partial closing of the holding section or the holder via the cover.

In some embodiments of the valve device described herein the second fluid line or a section thereof is inserted in the insert section.

In several embodiments, the outlet tap or a section thereof is inserted into the holding section of the valve device. Alternatively, the holding section is connected to the outlet tap.

In some embodiments, the second fluid line has a first free end for its connection to an effluent inlet line and a second free end for its connection to an effluent inlet opening of an effluent bag (or a fluid line connected thereto). The ends can be connectors, e.g., Luer-lock connector or of another type.

In several embodiments, the valve device described herein also has a switching element. The switching element is provided and arranged for manual—direct or indirect—switching of the blocking element from the first position to the second position and vice versa. Hereby, the switching element is preferably arranged such that when the locking element is manually switched from the first position to the second position, the actuator or a connecting section for connecting the switching element to the actuator is moved from the second setting to the first setting and vice versa.

In some embodiments the blocking element is arranged to be moved, when it rotates through a first angle α1, between a second position, in which the second fluid line is completely released from the blocking element, and a first position in which the second fluid line is completely closed by the blocking element. Herein the actuator may be arranged to be moved, when it rotates through a second angle α2, between a first setting, in which the effluent outlet line is completely closed by the actuator, into a second setting, in which the actuator allows a flow for the first time through the effluent outlet line and is therefore set open, whereby the first angle α1 is smaller than the second angle α2.

In several embodiments, the effluent outlet line is in conveying connection with at least one pump and/or a pump drive of a pump.

In some embodiments, the set described herein further includes an effluent bag for receiving effluent resulting from a blood treatment. The effluent bag for its part includes an effluent inlet opening and an effluent outlet opening separate therefrom, as well as an outlet tap for closing the effluent outlet opening, or an attachment for the outlet tap. The valve device is preferably configured to hold the outlet tap, preferably in a form-fitting and/or force-fitting manner, via its holding section.

In several embodiments, the pump or the pump drive has at least one magnetically mounted or driven pump section, in particular a pump head, in the effluent outlet line. This pump section or pump head is designed, for example, as an impeller pump head or as its rotor.

In several embodiments, the blood treatment apparatus described herein is embodied as a hemodialysis apparatus, hemofiltration apparatus or hemodiafiltration apparatus, in particular as an apparatus for use in continuous venous hemodiafiltration (CVV-HDF) and/or for use in acute dialysis.

In some embodiments, the blood treatment apparatus optionally includes a charging station for a voltage source for the pump drive of the pump. The voltage source can be a low voltage or low current source.

In several embodiments, the effluent bag can be any type of container, for example a container with a flexible outer skin such as a film, or made of film, a container with a hard outer skin, or a hard outer skin such as a canister, etc.

In certain embodiments, the outlet tap is actuated in such a way that a fluid connection which exists between the interior of the effluent bag and the interior of the effluent outlet line is interrupted.

In several embodiments, the outlet tap has an electrically insulating effect. This can preferably be understood to mean that fluids that can enter into the outlet tap via a connector or connection of the outlet tap (such as the effluent inflow into the effluent bag) and each surface with which they thereby come into contact, cannot come into contact with other fluids which can enter into the latter via another connector or connection of the outlet tap (such as the effluent outflow out of the effluent bag) and any surfaces with which they come into contact, cannot come into contact with each other due to the transmission of an electrical current and/or do not transmit current.

In some embodiments, the second fluid line can be inserted at the factory, i.e., the second fluid line is already inserted upon delivery and secured against removal, for example by connecting or snapping two components, in particular housing parts, e.g., the rotary bolt and the holder, with each other, wherein the connecting or snapping prevents a subsequent removal of the second fluid line from the valve device.

In some embodiments, the second fluid line cannot be removed from the valve device, at least not in a non-destructive manner, for example through an design with an undercut.

In certain embodiments, it can be provided that the actuation of the switching element is only possible when a second fluid line is actually inserted into the insert section of the valve device. This can be implemented, for example, via a pin on which the second fluid line presses. If the pin is not pressed by the second fluid line, then the switching lever can preferably not be actuated.

Several or all of the embodiments may include one or several or all of the advantages mentioned above and/or in the following.

In some embodiments, it is proposed that either the effluent inlet line or the effluent outlet line is interrupted in an insulating manner at any point in time. This advantageously reliably ensures that at no point in time is there a conductive connection between the effluent inlet line and the drain, i.e., the patient is never grounded against the drain. This advantageously avoids the risk that, when the contents of the effluent bag are drained, an electrically conductive contact occurs between the liquid and the earth, so that the permissible patient leakage currents would be exceeded. This contributes advantageously to increasing patient safety.

An advantage can be that with the effluent bag described herein, other than is the case with conventional effluent bags, if the effluent bag is to remain on the weighing device of the treatment device when it is emptied, the effluent inlet line does not, for safety reasons have to be separated manually from the effluent bag in order to ensure fluidic separation of the bag or its contents from the patient. Nevertheless, even if the effluent bag remains attached to and connected to the treatment device, a risk to the patient due to electrical currents can be excluded.

Another advantage is that implementing the described systems, methods, and devices is relatively straightforward.

One advantage that can be achieved via certain embodiments is that both actions, namely both the filling and emptying of the effluent bag, are coupled to one another by integrating the components to be operated for this purpose into a single component and are thus connected to one another for joint operation. The forced coupling helps ensure that manual steps associated with emptying are not forgotten. In addition, manual or automatic steps are saved.

The flow paths between the drain on the one side and the dialyzer or patient on the other side can advantageously be electrically insulated from one another, which ensures the safety of the patient.

It is also advantageous that the valve device can be provided in order to be operated manually. Therefore, in many embodiments, the methods, systems, and devices described herein do not require any intervention in the control or regulation of the blood treatment apparatus and therefore also advantageously allows cost-effective retrofitting of existing systems.

### BRIEF DESCRIPTION OF THE DRAWINGS

In the following, the present disclosure is described purely by way of example with reference to the attached figures. In them, the same reference symbols denote the same or the same components. The following applies:

FIG. 1 shows in a simplified representation a blood treatment apparatus having an extracorporeal blood circuit in an exemplary embodiment.

FIG. 2a shows in a simplified representation, a plurality of lines and the function of a set having an indicated valve device and an effluent bag in a state in which the effluent can be supplied to the effluent bag via the effluent inlet line.

FIG. 2b shows, in a simplified representation, the lines and the set of FIG. 2a in a state in which the effluent bag can be emptied via the effluent outlet line.

FIG. 3 shows in a schematically simplified representation, an effluent bag in a first embodiment having a valve device, which is shown in a second embodiment.

FIG. 4 shows in a top view, the valve device of FIG. 3, enlarged.

FIG. 5a shows in a schematically simplified representation, a third embodiment of a valve device, partly in a cross section, together with an outlet tap in an unassembled state, in which the outlet tap is not inserted in the valve device.

FIG. 5b shows in a schematically simplified representation the valve device of FIG. 5a, partly in a cross section, together with the outlet tap in an assembled state, in which the outlet tap is inserted in the valve device.

FIG. 6a shows the cross-sectional view of the valve device of FIG. 5a or 5b in the assembled state together with an outlet tap in a first state.

FIG. 6b shows a sectional view of the valve device of FIG. 6a in the assembled state together with an outlet tap in a second state.

FIG. 7a shows a top view of a blocking element of an exemplary valve device in a first position, in which a second fluid line which runs through the valve device is open.

FIG. 7b shows a top view of the blocking element of FIG. 7a in a second position, in which the second fluid line is closed/blocked.

FIG. 8a shows a top view of an outlet tap, on the left hand side from the outside, on the right hand side in an indicated longitudinal section, in which the effluent outlet line is closed.

FIG. 8b shows a top view of an outlet tap, to the right from the outside, on the left in an indicated longitudinal sectional representation, in which the effluent outlet line is open.

FIG. 9a shows a longitudinal section with a top view of a blocking element of the valve device in a further embodiment.

FIG. 9b shows a longitudinal section with a top view of an actuator of an outlet tap.

FIG. 10a shows a cross-section of a first embodiment of a blocking element of the valve device in its second position.

FIG. 10b shows the blocking element of FIG. 10a in its first position.

FIG. 11a shows a cross-section of a second embodiment of a blocking element of the valve device in its second position.

FIG. 11b shows the blocking element of FIG. 11a in its first position.

FIG. 12a shows an exemplary course of a method for preparing an effluent bag to receive effluent resulting from a blood treatment.

FIG. 12b shows an exemplary profile of a method for empting an effluent bag.

### DETAILED DESCRIPTION

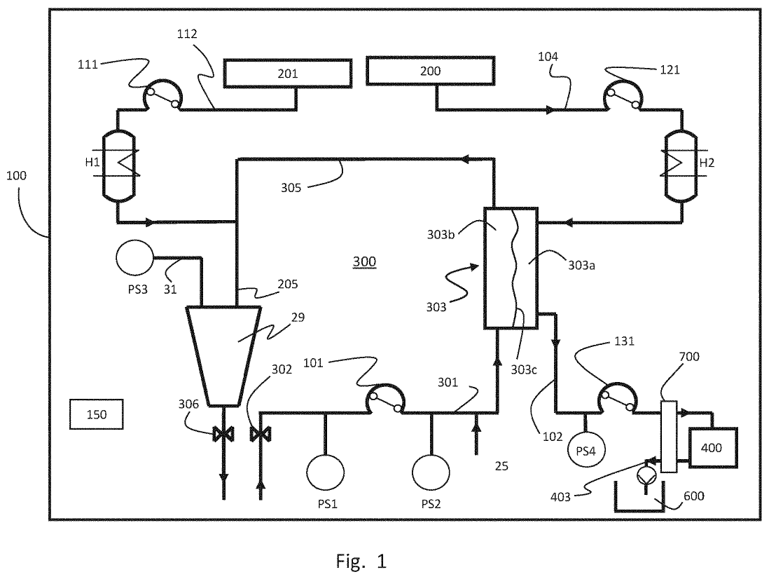

FIG. 1 shows in a highly simplified representation, a blood treatment apparatus 100 connected to a extracorporeal blood circuit 300.

The extracorporeal blood circuit 300 includes a first line 301, here in the form of an arterial line section.

The first line 301 is in fluid communication with a blood treatment device, here for example a blood filter or a dialyzer 303. The blood filter 303 includes a dialysis liquid chamber 303a and a blood chamber 303b, which are separated from each other by a mostly semi-permeable membrane 303c.

The extracorporeal blood circuit 300 further includes at least one second line 305, here in the form of a venous line section. Both the first line 301 and the second line 305 may be used to connect them to the vascular system of the patient, not shown.

The first line 301 is optionally connected to a (first) tubing clamp 302 for blocking or closing the line 301. The second line 305 is optionally connected to a (second) tubing clamp 306 for blocking or closing the line 305.

The blood treatment apparatus 100 represented in FIG. 1 only schematically and only by some of its devices includes a blood pump 101. During the patient's treatment, the blood pump 101 conveys blood through sections of the extracorporeal blood circuit 300 and towards the blood filter or dialyzer 303, as indicated by the small arrows, which in each of the figures generally indicate the direction of flow.

Using a pump for dialysis liquid 121, that may be designed as a roller pump or as any otherwise occluding pump, fresh dialysis liquid is pumped from a source 200 along the dialysis liquid inlet line 104 into the dialysis liquid chamber 303a. The dialysis liquid leaves the dialysis liquid chamber 303a as dialysate, possibly enriched by filtrate, towards the optional effluent bag 400 and is herein referred to as effluent.

The source 200 may be, for example a bag or a container. The source 200 may further be a fluid line, for example a hydraulic outlet or hydraulic port of the blood treatment apparatus 100 out of which on-line and/or continuously generated or mixed liquid is provided.

A further source 201 with substituate may be optionally provided. It may correspond to the source 200 or be a separate source.

An only roughly indicated control device or closed-loop control device 150 of the blood treatment apparatus 100 can be configured to initiate or carry out the method for controlling and/or regulating the treatment apparatus before, during and after treating a patient.

At the bottom right of FIG. 1 is roughly indicated that the valve device 700 intervenes in the effluent flow of the effluent inlet line 102 and the effluent outlet line 403 and enables or prevents the flow into the effluent bag 400 or out of the effluent bag 400. The mode of operation of the valve device 700 on the effluent supply line 102 and the effluent outlet line 403 as well as an effluent bag 400 are described in more detail in the following figures, as is also its possible, specific design and arrangement on the effluent bag 400 or its outlet tap 401.

In addition to the aforementioned blood pump 101, the arrangement shown in FIG. 1 further includes purely optionally a series of other pumps, in each case optional, namely the pump 111 for substituate, the pump 121 for dialysis liquid and the pump 131 for the effluent.

The pump 121 is provided to supply dialysis liquid out of a source 200, for example a bag, via an optional existing bag heater H2 having a bag, to the blood filter 303 via a dialysis liquid inlet line 104.

The thus supplied dialysis liquid exits from the blood filter 303 via a dialysate outlet line 102, supported by the pump 131, and may be discarded (see above).

Upstream of blood pump 101 an optional arterial sensor PS1 is provided. It measures the pressure in the arterial line during the patient's treatment.

Downstream of the blood pump 101, but upstream of the blood filter 303 and, if provided, preferably upstream of an addition point 25 for an anticoagulant, for example heparin, a further optional pressure sensor PS2 is provided. It measures the pressure upstream of the blood filter 303 (“pre-hemofilter”).

Again, a further pressure sensor may be provided as PS4 downstream of the blood filter 303, however preferably upstream of the pump 131, in the dialysate outlet line 102 in order to measure the filtrate pressure of the blood filter 303.

Blood, which leaves the blood filter 303, passes through an optional venous blood chamber 29, which can include a ventilation device 31 and may be in fluid communication with a further pressure sensor PS3.

In the exemplary arrangement shown in FIG. 1 the control device or closed-loop control device 150 can be in cable or wireless signal communication with any of the components referred to herein—especially or in particular the blood pump 101—in order to control or regulate the blood treatment apparatus 100. It is optionally configured to carry out the method described herein.

The optional pump 111 is provided in order to supply substituate from the optional source 201, for example a bag, and via an optionally present bag heater H1 having a bag to the second line 305.

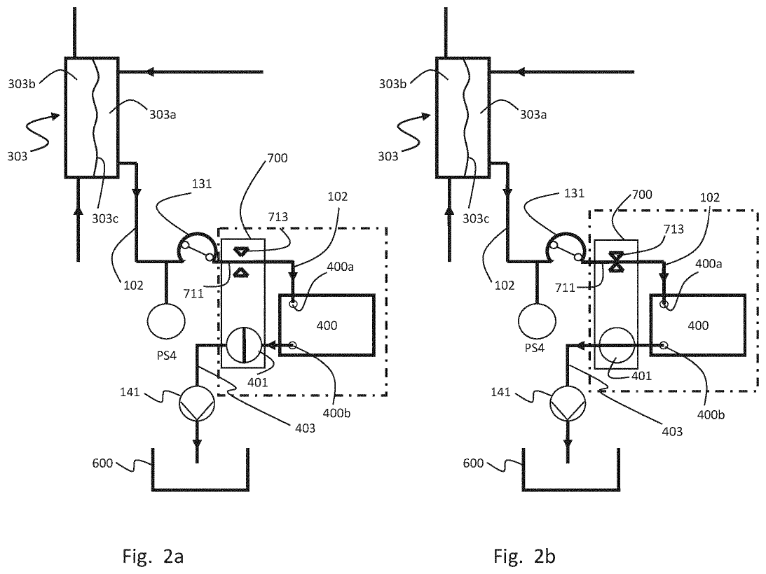

FIG. 2a shows in a simplified representation a plurality of lines and the function of a set which is arranged downstream of the blood filter 303 on a blood treatment apparatus 100.

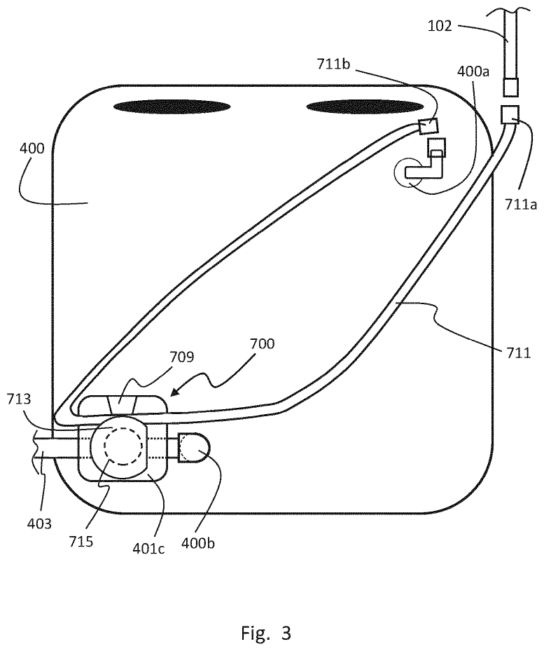

The set includes a valve device 700 and a second fluid line 711, which may be understood to be an extension of the effluent inlet line 102 (see FIG. 3).

Further, the set includes an effluent bag 400 for receiving effluent which result from a blood treatment using the blood treatment apparatus 100. The effluent bag for its part includes an effluent inlet opening 400a and an effluent outlet opening 400b, the effluent outlet opening 400b including an outlet tap 401 or a fluidic connection to such.

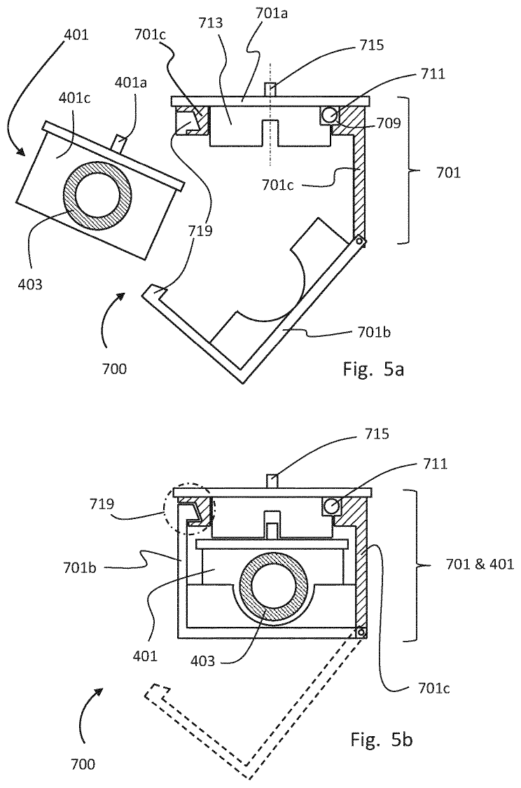

The valve device 700 is configured to hold the outlet tap 401 using a holding section 701 (see FIG. 5a and FIG. 5b), preferably in a form-fitting and/or force-fitting manner.

Moreover, the arrangement of FIG. 2a shows an effluent pump, also referred to as a filtrate or dialysate pump, which serves to convey effluent starting from a dialysis liquid chamber 303a of the blood filter 303 towards the effluent inlet opening 400a of the effluent bag 400. The effluent is first collected in the effluent bag 400 and then after the treatment or at so-called empty bag intervals, it is discarded, e.g., via an optional further pump 141 or through gravity via the effluent outlet opening 400b and via the effluent outlet line 403 into an optional drain 600.

The example in FIG. 2a shows the set at a point at which effluent is supplied from the dialysis liquid chamber 303a of the blood filter 300 to the effluent bag 400 via the effluent pump 131 in the effluent inlet line 102.

The outlet tap 401, which is suitable for switching between two settings, is arranged between the pump 141 and the effluent bag 400. In the setting shown, it does not allow any flow of liquid through the effluent outlet line 403. A blocking element 713 within the valve device 700 upstream of the effluent bag 400 is in a second position in which the second fluid line 711 is open. In the arrangement at the point shown in FIG. 2a, the effluent bag 400, arranged on the blood treatment apparatus 100, is filled with effluent, but not emptied, since no effluent can flow off via the closed effluent outlet line 403.

The pump 141, which is arranged in the effluent outlet line 403, may optionally include or consists of at least a pump drive and a pump head, (not shown in the figures). In the setting in FIG. 2a it is not in operation (“OFF”), as the effluent outlet line 403 is not guiding any effluent which could be discharged into the drain 600 by the pump 141.

In any embodiment, the pump 141 can be a roller pump. Alternatively, it is not a roller pump in any embodiment. In several embodiments the pump 141 is an impeller pump in others it is not.

FIG. 2b shows in simplified representation the exemplary set of FIG. 2a.

Everything which applies to the set in FIG. 2a applies analogously to FIG. 2b.

The arrangement of FIG. 2b shows the set at a moment during which effluent is conveyed out of the effluent bag 400 by the pump 141 via the effluent outlet line 403 and is discarded, for example into an optional drain 600.

In the setting shown, the outlet tap 401 allows a flow of liquid through the effluent outlet line 403. A blocking element 713 upstream of the effluent bag 400 is in a first position, i.e., the second fluid line 711 is closed, therefore the flow cannot flow through the blocking element 713.

In the setting or position shown in FIG. 2b, the (filled) effluent bag 400, arranged on the blood treatment apparatus 100, is emptied using the pump 141 via the now opened effluent outlet line 403.

In the setting in FIG. 2b, the pump 141 is in operation (“ON”), as the effluent outlet line 403 now guides effluent, which using the pump 141 can be discarded into the drain 600.

The present set advantageously couples the function of blocking element 713 and outlet tap 401 to each other. This is described in greater detail in the following figures.

FIG. 3 shows a schematically simplified representation of an effluent bag 400 in a first embodiments having a valve device 700, also in a first embodiment.

The effluent bag 400 has an effluent inlet opening 400a and an effluent outlet opening 400b. The effluent inlet opening 400a is suitable for fluid connection to the dialysate outlet line or effluent inlet line 102 via suitable connectors. The effluent outlet opening 400b is suitable for fluidic connection to the effluent outlet line 403 using suitable connectors.

The effluent outlet line 403 includes an outlet tap 401, which is suitable for allowing or preventing a flow of liquid in the effluent outlet line 403. For this purpose, the effluent outlet line 403 has an actuator 401c (see FIG. 5a), which can be set to a first setting and to a second setting. In the first setting the flow out of the effluent outlet opening 400b along the effluent outlet line 403 is blocked, in the second setting flow out of the effluent outlet opening 400b along the effluent outlet line 403 and thus out of the effluent bag 400 is open.

The valve device 700 can be held on or connected to the outlet tap 401 by a holding section 701.

A second fluid line 711 can be inserted into an insert section 709 of the valve device 700. The second fluid line 711 acts as an “extension” of the effluent inlet line 102 by connecting a first free end 711a to the effluent inlet line 102, while the second free end 711b of the second fluid line 711 is connected to the effluent inlet opening 400a or to a piece of tubing provided thereon.

The valve device further includes a blocking element 713 which can be switched between a first position and a second position. In the first position, the blocking element 713 prevents liquid from flowing through the second fluid line 711, and in the second position it allows liquid to flow through the second fluid line 711.

The actuator 401c and blocking element 713 are coupled or connected in such a way that the blocking element 713 is positively guided into the second position when the actuator 401c is brought into the first setting and that the blocking element 713 is positively guided into the first position when the actuator 401c is brought into the second setting.

This results in two states I or II of the outlet tubing system, which can be found in the following table:

| | actuator | blocking-element | second line | Effluent-outlet- |
|---|---|---|---|---|
| State | 401c | 713 | 711 | line 403 |
| I | 1. setting | 2. position | open | closed |
| II | 2. setting | 1. position | closed | open |

Here, state I is the state in which the effluent bag 400 is filled (see also FIG. 2a) and state II is the state in which the effluent bag 400 is emptied (see also FIG. 2b).

The example in FIG. 3 shows state II of the valve device 700, whereby the actuator 401c and the effluent outlet line 403 within the valve device are only indicated using dashed lines. In addition, the second fluid line 711 would still have to be connected by its ends 711a and 711b to the effluent inlet line 102 or the effluent inlet opening 400a, respectively, in order to obtain a functional set.

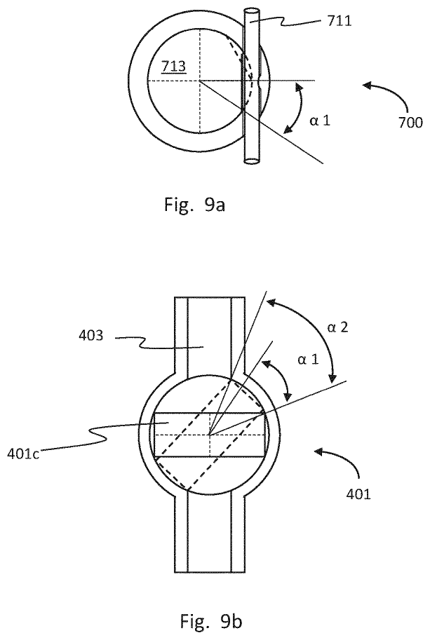

When a switching element 715 is actuated clockwise or counterclockwise, the valve device 700 is, in each case, guided from the first state I into the second state II, or vice versa. This changes both the setting of the actuator 401c and the position of the blocking element 713. The actuation of the switching element 715 is preferably a rotation of 90° around its center point, by which both the blocking element 713 and the actuator 401c each positively guided by the same angle. In the transition between the two states I and II, it should preferably be ensured that when the actuator 401c moves from the first setting to the second setting or when the blocking element 713 moves from the second position to the first position, and vice versa, at a certain angle, the vertex of which lies in the center point or on a longitudinal axis of the actuator or the blocking element, advantageously in a certain angular range, both the second fluid line 711 and the effluent outlet line 403 are interrupted, i.e., in that liquid cannot flow through either of the two lines 711, 403 (see FIGS. 9a and 9b). In this way, impermissible patient leakage currents can be safely avoided.

The switching element 715 is only indicated in FIG. 3 by a dashed circle.

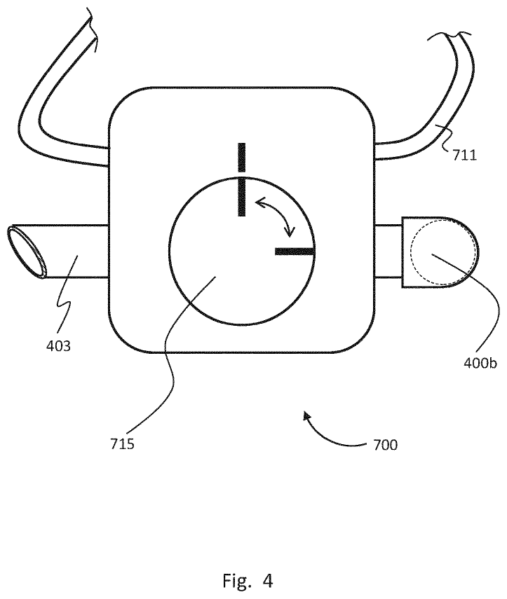

FIG. 4 shows an enlarged exemplary top view or an external view of the valve device 700 of FIG. 3.

The second fluid line 711 can be seen, shown in section, which is inserted into the valve device 700, and the effluent outlet line 403, which leads through the valve device 700 at the bottom and is connected to the effluent outlet opening 400b.

Furthermore, the switching element 715 can be seen, which is arranged rotatably on the upper side of the valve device 700. the switching element 715 is used to manually switch the valve device 700 from state I to state II, as set out herein, and vice versa.

FIG. 5a shows, in a schematically simplified representation, a set in an exemplary embodiment.

The set includes the valve device 700, which in turn has a holding section 701. The holding section 701 in turn has a rotary bolt 701a, a holder 701c, and a cover 701b. The rotary bolt 701a can be inserted into an opening of the holder 701c.

The set further includes a second fluid line 711, which is received or inserted into an insert section 709 of the valve device 700 and by connecting the holder 701c to the rotary bolt 701a it is secured against removal. Securing against removal can be implemented by designing the holder 701c with an undercut, in which the rotary bolt 701a can snap into the holder 701c radially on the inside or outside, preferably rotatably. The insert section 709 with the inserted second fluid line 711 can be seen at the top right in FIG. 5a. The fluid line 711 is shown in a cross section with a view of the cut surface and a view into its lumen.

FIG. 5a shows the valve device 700 and outlet tap 401 in a state of assembly in which the second fluid line 711 has already been inserted, but the holding section 701 is not yet connected to the outlet tap 401. The outlet tap 401 can be seen as a separate component on the left in FIG. 5a.

A section of the effluent outlet line 403 can clearly be seen in a cross section, which leads through the outlet tap 401, with a view of the cut surface and into its lumen.

On top of the outlet tap 401 is a grip section 401a, which can function as a coupling section or connecting section to the holding section 701 of the valve device 700. When used as intended, it serves to manually move the actuator 401c from its first setting to its second setting, and vice versa.

The rotary bolt 701a has a switching element 715 in the upper center, by which the, by then, installed valve device 700 can be guided manually between state I and state II and vice versa, during the treatment. For a description of these states, reference is made to the preceding statements.

The cover 701b can be moved from an open cover setting (shown in FIG. 5a) into a closed cover setting (see FIG. 5b). In the open cover setting, the outlet tap 401 can be inserted into the cover 701b. The grip section 401a of the outlet tap 401 can be pushed into a recess in the blocking element 713 or otherwise connected to the blocking element 713. The blocking element 713 can in turn be connected to the rotary bolt 701a in a rotationally stable manner or can be integral with it.

Cover 701b and holder 701c can be connected or are connectable to each other using a hinge section and/or a snap or click system 719. The snap or click system 719, or an alternative device, can hold cover 701b and holder 701c in the second, closed cover setting.

The outlet tap 401 is thus inserted into the cover 701b for its connection to the valve device 700. When the cover 701b is closed, a bar 401b (FIG. 8a) engages in the blocking element 713. Cover 701b and holder 701c surround the outlet tap 401. For this purpose, cover 701b and/or holder 701c may have suitable recesses which can, for example, reproduce the outer shape of outlet tap 401.

FIG. 5b shows in a schematically simplified representation the set of FIG. 5a.

The cover 701b is closed and the outlet tap 401 is received or inserted into the valve device 700. The example in FIG. 5b can also be referred to as in an assembled state.

In this example, the second fluid line 711 is completely occluded when the rotary bolt 701a is rotated using the blocking element 713 due to its radial compression in sections.

In the closed cover setting, which in the example of FIGS. 5a and 5b, is secured purely by way of example using a tongue and groove connection, the outlet tap 401 is securely held in the holder 701c and coupled to the blocking element 713 in the manner described above in a rotational connection. Other suitable devices for closing the cover 701b, such as clip or clamp devices, are also encompassed.

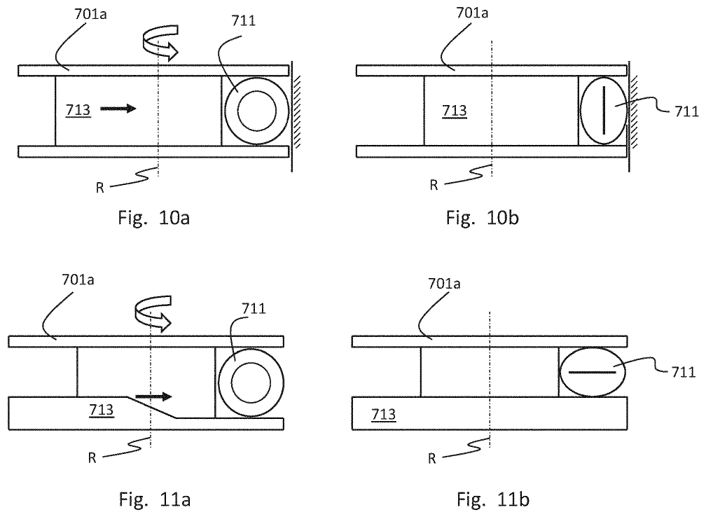

Other mechanical solutions of the blocking element and/or couplings of the same with further components, which can close the second fluid line 711, are likewise encompassed by the present disclosure. These can be suitable for closing the second fluid line 711, for example radially or axially, or for completely occluding it. The set described herein also encompasses translational mechanisms as blocking elements suitable for this purpose. In this regard, reference is also made to the description of FIGS. 10 and 11.

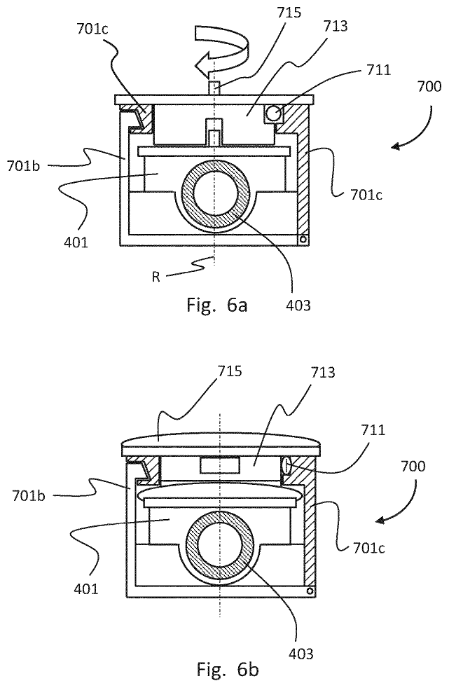

FIG. 6a shows a sectional view of a valve device 700 in a further embodiment with an outlet tap 401 received therein, in a (first) state I.

The lumen of the second fluid line 711 is open in the area of the valve device 700, whereas the lumen of the effluent outlet line 403 is closed by the outlet tap 401. The “blocking” of the effluent outlet line 403 cannot be seen from this view, since this only allows a view of the line inlets of the outlet tap 401. The blocking is explained in more detail in FIGS. 8a and 8b.

In this state I, the effluent bag 400 (see preceding figures) could be filled.

A rotation axis R, which blocking element 713 and actuator 401c can be rotated around by actuating the switching element 715, which is preferably provided in a rotationally fixed manner, is indicated in FIG. 6a and in the following figures by a dash-dot line.

FIG. 6b shows an illustration in section of the valve device 700 of FIG. 6a with an outlet tap 401 received therein in a (second) state II.

The switching element 715 was rotated through an angle of preferably 90° and is now visible from the side.

The second fluid line is closed using the blocking element 713, while the effluent outlet line 403 has been opened by changing the setting of the actuator 401c or a flow has been made possible (again).

The blocking of the second fluid line 711 can be clearly seen in this view.

The effluent bag 400 (see previous figures) would be able to be emptied in this state II.

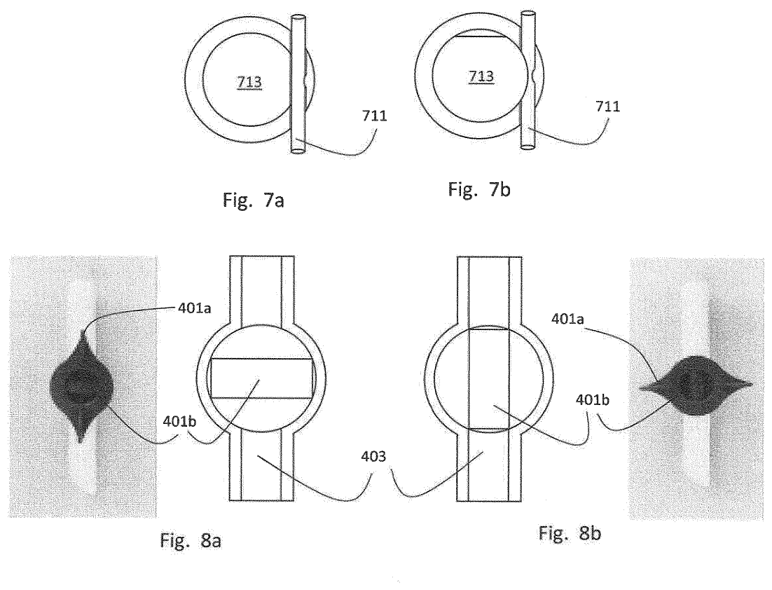

FIG. 7a shows a top view of a blocking element 713 of the valve device 700 in a first state I.

The blocking element 713 is in its second position. Due to the geometric configuration of the blocking element 713, in this position it releases a second fluid line 711. Effluent can flow through it.

FIG. 7b shows a top view of the blocking element 713 of FIG. 7 in a second state II.

The blocking element 713 is in its first position. Due to the geometric design of the blocking element 713, it completely occludes the second fluid line 711 in this position, e.g., an electrically conductive liquid column, one which is present during use in the second fluid line 711, is interrupted in an electrically insulating manner. Therefore, effluent cannot flow through the second fluid line 711; current cannot flow over a stationary or flowing, continuous column of effluent across the occluded section of the second fluid line 711.

FIG. 8a is to be seen in connection with FIG. 7a and shows a top view of an actuator 401c of an outlet tap 401 which would be inserted into the aforementioned holder 701c of the valve device 700. On the left, the outlet tap 401 is shown from the outside, on the right, in a longitudinal sectional view.

The outlet tap 401 is in the first state I, i.e., the effluent outlet line 403 is blocked. Therefore, if the effluent bag 400 is filled, effluent could not drain from it.

In the external view on the left, a grip section 401a of the outlet tap 401 can be seen, which can serve as a connecting section to the rotary bolt 701a and would forcibly rotate when the switching element 715 was rotated. The grip section 401a is perpendicular to a bar 401b, the setting of which can reveal from outside the outlet tap 401 whether the effluent outlet line 403 is open or closed. In some embodiments, the bar can also function as a coupling section between outlet tap 401 and valve device 700.

Alternatively or additionally, in several embodiments the grip section 401a of the outlet tap 401 can also serve as a connecting section to the locking element 713 in order rotate with it by force. This can be done, for example, by inserting the grip section 401 into a groove on the underside of the blocking element 713.

FIG. 8b is to be seen in connection with FIG. 7b and shows a top view of the actuator 401c of FIG. 8a in a second setting. To the right, the outlet tap 401 is shown from the outside to the left, in a longitudinal sectional view.

The outlet tap 401 is in the second state II, i.e., the effluent outlet line 403 is open, therefore the effluent bag 400 can be emptied, as effluent can now drain from it.

In the external view on the left it can be seen that the grip section 401a is now horizontal, that is to say rotated in a position 90° to the position in FIG. 8a. In a connection with a rotary bolt 701a, this rotation would have brought about a forced guidance of the blocking section 713, which in turn would have caused the blocking of the second fluid line 711 (see FIG. 7b).

FIG. 9a shows a sectional view from above of an exposed blocking element 713 of the valve device 700 in a first state I of the valve device 700. The blocking element 713 is in its second position, i.e., effluent can flow towards the effluent inlet opening 400a through the second fluid line 711.

As the blocking element 713 rotates by actuating the switching element 715 of the valve device 700 counterclockwise in order to move the valve device 700 to its second state II, the second fluid line 711 is blocked due to the geometric design of the blocking element 713 precisely from the moment when the blocking element 713 is rotated by at least the angle α1.

FIG. 9b can be seen in connection with FIG. 9a and shows a sectional view from above of an actuator 401c of an outlet tap 401 in the first state I. It is comparable to the state I of FIG. 9a. The actuator 401c is in its second setting.

As the actuator 401c rotates counter-clockwise, positively guided by coupling its rotation to an actuation of the switching element 715 of the valve device 700, analogous to the description of FIG. 9a, the effluent outlet line 403 remains blocked until it has been rotated through an angle α2. Only after continuing to rotate counter-clockwise is the effluent outlet line 403 released and effluent can flow through it towards the drain 600 (see FIGS. 1 to 2b). This allows the effluent bag 400 to be emptied.

As already indicated with regard to FIG. 3, the valve device 700 should preferably ensure that during the transition between state I and state II the actuator 401c and the blocking element 713 are in such a position in relation to each other that both the second fluid line 711 and the effluent outlet line 403 are interrupted, i.e., liquid cannot flow through either of the two lines 711, 403. This is the case if it has been structurally achieved, e.g., through geometric design or via advantageous dimensioning, that the rotation must be greater in order to open one line than it is to close the other. When in use, the liquid column is interrupted in state II due to the occlusion of line 711, which is the reason for the electrical insulation along the —now interrupted—liquid column.

This is the case in the present example of FIGS. 9a and 9b, when α1<α2 applies. When setting the switching element 715 of the valve device 700 in the angular range between α1 and α2, it can be ensured that liquid cannot flow through either of the two lines 711 and 403. In this way, impermissible patient leakage currents can be safely avoided.

FIG. 10a shows a cross-section of a first embodiments of a blocking element 713 of the valve device 700 in its second position, i.e., with the second fluid line 711 open, through which in this position effluent can flow.

The blocking element 713 is intended to be rotated around the rotation axis R, indicated by a curved arrow around the rotation axis R, in order to move the valve device 700 from a state I to a state II, i.e., to occlude the second fluid line 711.

FIG. 10b shows the blocking element 713 of FIG. 10a in its first position, i.e., with the second fluid line 711 fully occluded.

The blocking element 713 is geometrically designed in such a way that, by rotating the blocking element starting from its second position in FIG. 10a, preferably by an angle of approx. 90°, the second fluid line 711 is completely occluded by squeezing it together in sections, thus interrupting the electrically conductive fluid column in the second fluid line 711 in an electrically insulating manner. This is done in a radial direction, relative to the blocking element 713.

FIG. 11a shows a cross-section of a second embodiment of a blocking element 713 of the valve device 700, the blocking element 713 being in its second position, i.e., with the second fluid line 711 open, through which effluent can flow in this position.

Analogous to FIG. 10a, the blocking element 713 should then be rotated around the rotation axis R, indicated by a curved arrow around the rotation axis R, in order to move the valve device 700 from a state I to a state II, i.e., to occlude the second fluid line 711.

FIG. 11b shows the blocking element 713 of FIG. 11a in its first position, i.e., with the second fluid line 711 fully occluded

The blocking element 713 is geometrically designed, in its lower area in the example of FIG. 11b, in such a way that by rotating the blocking element starting from its second position in FIG. 11a, preferably by an angle of approx. 90°, the second fluid line 711 is completely occluded by squeezing it together in sections and thus the electrically conductive liquid column in the second fluid line 711 is interrupted in an electrically insulating manner. This is done in the axial direction, relative to the blocking element 713.

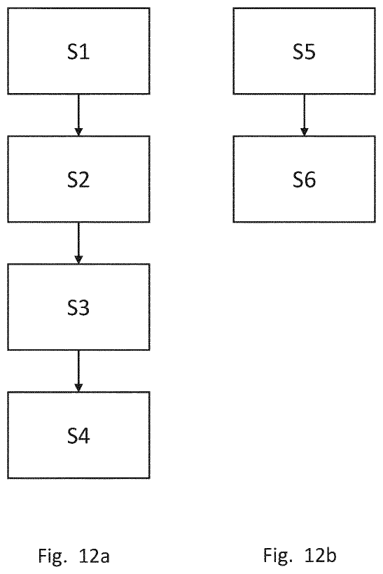

FIG. 12a shows an exemplary course of a method for preparing an effluent bag 400 (see FIG. 3) to receive the effluent resulting from a blood treatment, the method including:

S1 represents providing of an effluent bag 400 or a set as described herein.

S2 represents a connection of the second fluid line 711 both to the effluent inlet line 102 and to the effluent inlet opening 400a. Suitable connectors are provided at the free ends 711a, 711b of the second fluid line 711, for this purpose.

S3 represents an insertion of a tubing section of the second fluid line 711 into an insert section 709 of the valve device 700, provided that this has not already been done prior to the method or during the manufacture of the valve device 700.

S4 represents a connection of the valve device 700 to the outlet tap 401 via the holding section 701.

The outlet tap 401 of the effluent bag 400, which was prepared using the method, is connected to the valve device 700 in such a way so that when a switching element 715 of the valve device 700 is actuated, both the blocking element 713 and the actuator 401c of the outlet tap 401 are forcibly carried along (see preceding figures).

FIG. 12b shows an exemplary course of a method for emptying an effluent bag 400.

It encompasses:

S5 as providing an effluent bag 400 or a set as described herein, in particular prepared according to the exemplary method in FIG. 12a; and

S6 as an actuating of the switching element 715 of the valve device 700 in such a way that a fluid connection is established between the inside of the effluent bag 400 and the inside of the effluent outlet line 403.

The valve device 700, connected to the outlet tap 401, according to the method described with regard to FIG. 12a, preferably ensures that during an upcoming blood treatment, effluent can flow through either the second fluid line 711 or the effluent outlet line 403 (see FIG. 10b). It can be switched between the above-mentioned states I and II via the switching element 715, i.e., it can be switched back and forth so that always exactly one or neither of the two lines is open.

The valve device 700 also ensures that always either the liquid column of the second fluid line 711 due to its occlusion in state II is interrupted in an electrically insulating manner or the liquid column in the effluent outlet line 403 in state I is interrupted in an electrically insulating manner via the closed outlet tap 401 so that the patient cannot be electrically earthed either in state I nor in state II along the effluent with the drain 600.

This means that an effluent bag 400 can either be filled using the second fluid line 711 or emptied using the effluent outlet line 403, but never both at the same time.

### LIST OF REFERENCE NUMERALS

25 addition site for heparin (optional)
29 venous blood chamber (optional)
31 ventilation or aeration device
100 blood treatment apparatus
101 blood pump
102 dialysate outlet line, effluent inlet line
104 dialysis liquid inlet line
111 pump for substituate
121 pump for dialysis liquid
131 pump for dialysate or effluent in effluent inlet line
132 connector
141 pump in effluent outlet line
150 control device or closed-loop control device
160 tubing clamp in effluent inlet line
200 dialysis liquid source
201 substituate source, optional
300 extracorporeal blood circuit
301 first line (arterial line section)
302 (first) tubing clamp
303 blood filter or dialyzer
303a dialysis liquid chamber
303b blood chamber
303c semi-permeable membrane
305 second line (venous line section)
306 (second) tubing clamp
400 effluent bag
400a effluent inlet opening
400b effluent outlet opening
401 outlet tap
401a grip section
401b bar
401c actuator or adjustment element
10 403 effluent outlet line
600 drain
700 valve device
15 701 holding section
701a rotary bolt
701b cover or top panel
701c holder
709 insert section for the second fluid line
711 second fluid line
711a first free end
711b second free end
713 blocking element
25 715 switching element
717 switching lever
719 snap or click system
H2 bag heater with bag (dialysis liquid)
H1 bag heater with bag (substituate)
PS1, PS2 arterial pressure sensor (optional)
PS3 pressure sensor (optional)
PS4 pressure sensor for measuring filter pressure
R rotation axis
S1, . . . , S6 method steps
α1 first angle
α2 second angle

1-17. (canceled)

18. A valve device for connecting to an outlet tap, the outlet tap comprising an actuator being arranged in or on an effluent outlet line or an attachment therefor, associated with an effluent outlet opening of an effluent bag for receiving effluents resulting from a blood treatment, wherein the actuator of the outlet tap has a first setting, in which the flow from the effluent outlet opening along the effluent outlet line is blocked, and a second setting, in which the flow is released from the effluent outlet opening along the effluent outlet line and out of the effluent bag, wherein the valve device comprises:
a holding section for holding the valve device at or on the outlet tap;
an insert section for inserting a second fluid line into the valve device; and
a blocking element which can be switched between at least a first position and a second position;
wherein, when the blocking element is arranged in the first position, the blocking element acts directly or indirectly on the second fluid line to block flow along the second fluid line, and when the blocking element is arranged in the second position, the blocking element allows flow along the second fluid line.

19. The valve device according to claim 18, wherein when the blocking element is arranged in the first position, the blocking element interrupts an electrically conductive liquid column in the second fluid line in an electrically insulating manner.

20. The valve device according to claim 18, wherein the blocking element is arranged such that, as the actuator is being moved into the first setting the blocking element is forcibly being moved into the second position, and as the actuator is being moved to the second setting, the blocking element is forcibly being moved to the first position, and/or vice versa.

21. The valve device according to claim 20, wherein the blocking element is arranged such that, when the actuator is moved into the second setting, the blocking element maintains a position in which the flow along the second fluid line is or remains prevented.

22. The valve device according to claim 21, wherein an electrically conductive liquid column in the second fluid line is interrupted in an electrically insulating manner before the actuator assumes the second setting.

23. The valve device according to claim 18, wherein the holding section comprises a rotary bolt with a cover, wherein the cover is movable between a first, open cover-setting for inserting or removing the outlet tap from the holding section or the rotary bolt and a second cover-setting for force-fitting and/or form-fitting of the outlet tap in the holding section.

24. The valve device according to claim 18, wherein the holding section has a rotary bolt for receiving the outlet tap or a section thereof and a cover, wherein the rotary bolt and/or cover are designed with a snap or click system for their joint or mutual connection and/or for at least partially closing the rotary bolt using the cover.

25. A valve device according to claim 18, wherein the second fluid line or a section thereof is arranged in the insert section.

26. The valve device according to claim 18, wherein the outlet tap, or a section thereof, is arranged in the holding section, or wherein the holding section is connected to the outlet tap.

27. The valve device according to claim 26, wherein the second fluid line comprises a first free end for connecting it to an effluent inlet line and a second free end for connecting the second fluid line to an effluent inlet opening of an effluent bag or a fluid line connected thereto.

28. The valve device according to claim 18, further comprising a switching element, arranged for manual switching of the blocking element from the first position to the second position.

29. The valve device according to claim 28, wherein the switching element is arranged such that in manual switching of the blocking element from the first position to the second position, the actuator or a connecting section for connecting the switching element to the actuator is moved from the second setting to the first setting.

30. The valve device according to claim 18, wherein when the blocking element rotates through a first angle, the blocking element is arranged to be moved between the second position, in which the second fluid line is completely released from the blocking element, and the first position in which the second fluid line is completely closed by the blocking element, wherein when the actuator rotates through a second angle, the actuator is arranged to be moved between the first setting, in which the effluent outlet line is completely closed by the actuator, into the second setting, in which flow is allowed for the first time in the effluent outlet line via the actuator, wherein the first angle is smaller than the second angle.

31. An effluent bag for receiving effluent resulting from a blood treatment, the effluent bag comprising:
an effluent inlet opening;
an effluent outlet opening;
an outlet tap connected to the effluent outlet opening, the outlet tap comprising an actuator and arranged in or at an effluent outlet line, wherein the actuator of the outlet tap has a first setting, in which the flow from the effluent outlet opening along the effluent outlet line is blocked, and a second setting in which the flow is released from the effluent outlet opening along the effluent outlet line and out of the effluent bag; and
a valve device according to claim 18.

32. A set having a valve device according to claim 18 and a second fluid line for inserting into the insert section of the valve device.

33. The set according to claim 32, further comprising an effluent bag for receiving effluents resulting from a blood treatment, the effluent bag comprising an effluent inlet opening and an effluent outlet opening as well as an outlet tap for closing the effluent outlet opening or comprises an attachment therefor.

34. A method for preparing an effluent bag for receiving effluents resulting from a blood treatment, the method comprising:
providing the set according to claim 32;
connecting the second fluid line to both the effluent inlet line and also to the effluent inlet opening using suitable connectors at free ends of the second fluid line;
inserting the second fluid line in the insert section of the valve device; and
connecting the valve device to the outlet tap using the holding section.

35. A method for emptying an effluent bag, the method comprising:
providing the set according to claim 32; and
actuating the switching element of the valve device in such a way that a fluid connection is established between the interior of the effluent bag and the interior of the effluent outlet line.

36. The method of claim 35, wherein providing the set comprises:
connecting the second fluid line to both the effluent inlet line and also to the effluent inlet opening using suitable connectors at free ends of the second fluid line;
inserting the second fluid line in the insert section of the valve device; and
connecting the valve device to the outlet tap using the holding section

37. A blood treatment apparatus connected to the set according to claim 32.

38. The blood treatment apparatus of claim 37, wherein the set is prepared by a method comprising:
connecting the second fluid line to both the effluent inlet line and also to the effluent inlet opening using suitable connectors at free ends of the second fluid line;
inserting the second fluid line in the insert section of the valve device;
connecting the valve device to the outlet tap using the holding section

39. The blood treatment apparatus according to claim 37, comprising a hemodialysis apparatus, hemofiltration apparatus or hemodiafiltration apparatus.

40. The blood treatment apparatus according to claim 39, comprising an apparatus for acute and chronic renal replacement therapy or for continuous renal replacement therapy (CRRT).
